(19) United States
(12) Patent Application Publication
(10) Pub. No.: US 2018/0132837 A1
(43) Pub. Date: May 17, 2018

(54) SHAPED DELIVERY SHEATH AND METHODS

(71) Applicant: W. L. Gore & Associates, Inc., Newark, DE (US)

(72) Inventors: Scot K. Mathena, Flagstaff, AZ (US); Richard L. McClure, Peoria, AZ (US)

(21) Appl. No.: 15/572,004

(22) PCT Filed: May 4, 2016
(86) PCT No.: PCT/US2016/030797

§ 371 Date: Nov. 6, 2017

Related U.S. Application Data
(60) Provisional application No. 62/157,062, filed on May 5, 2015.

(51) Int. Cl. A61B 17/00

(52) U.S. Cl. CPC A61B 17/00234; A61B 2017/00243; A61M 25/0041; A61B 2017/00623; A61B 2017/00867; A61B 2017/00331

(57) ABSTRACT

Embodiments relate to delivery systems for accessing a blood vessel. The delivery systems include a shaped accessory sheath having a default shape that may be temporarily altered to facilitate insertion through a patient's blood vessel and returned to the default shape after insertion to facilitate a medical procedure. Some of the present accessory sheaths are configured to have their default shapes altered via a dilator having a stiffness greater than that of a corresponding portion of the accessory sheath. Others of the present accessory sheaths are configured to have their respective default shapes altered by a shape memory material (SMM) that alters shape responsive to changes in certain condition(s) such as temperature.

### CROSS-REFERENCE TO RELATED APPLICATION

This application is a national phase application of PCT Application No. PCT/US2016/030797, filed May 4, 2016, which claims priority to Provisional Application No. 62/157,062, filed May 5, 2015, both of which are herein incorporated by reference in their entireties.

### FIELD

This disclosure relates to delivery sheaths and methods. More particularly, the disclosure relates to dilator and catheter introducer systems and methods for traversing blood vessels and organs.

### BACKGROUND

Catheter introducer systems are often introduced into blood vessels and organs for intraluminal diagnostics, treatment and delivery of medical devices and structures. The type of catheter introducer system utilized depends on the medical procedure performed, the route in the body taken, and individual patient anatomy, among other factors.

### SUMMARY

Embodiments relate to a shaped accessory sheath and related systems and methods for accessing a blood vessel or other portion of a patient's circulatory system. The shaped accessory sheath is shaped with or otherwise configured to adopt a default shape in the absence of a shape-altering force or structure such that the default shape may correspond to a desired location for a medical intervention within the patient's circulatory system. Some embodiments of the present systems include a dilator configured to alter the shape of at least a portion of the accessory sheath to facilitate insertion of an accessory sheath distal end to a desired location.

Some embodiments of the present delivery systems for accessing a blood vessel comprise: an accessory sheath comprising an elongated member having an accessory sheath body portion that includes an accessory sheath proximal end, an accessory sheath distal end opposite from the accessory sheath proximal end, and an accessory sheath lumen extending through the accessory sheath between the accessory sheath proximal end and the accessory sheath distal end, the accessory sheath including a curved section that is nearer the accessory sheath distal end than the accessory sheath proximal end; where a default shape of the curved section includes, from a proximal portion of the curved section and extending toward a distal portion of the curved section: (i) a first curved segment having a first radius of curvature; (ii) a second curved segment with a second radius of curvature that is smaller than the first radius of curvature and the curvature of the second curved segment is in another (e.g., different) direction than the curvature of the first curve; (iii) a third curved segment with a third radius of curvature that is smaller than the second radius of curvature and the curvature of the third curved segment is in a different direction than the curvature of the second curve; and (iv) a first axial segment between the third curved segment and the accessory sheath distal end; where the first curved segment and the second curved segment are disposed in a first plane, and the first axial segment is disposed in a second plane that is rotated relative to the first plane (e.g., around an axis that extends parallel to at least a portion of the accessory sheath between the accessory sheath proximal end and the curved section, which axis may, for example, be parallel to both of the first and second planes, and/or may be coaxial with an intersection of the first and second planes).

Some embodiments of the present delivery systems for accessing a blood vessel comprise: an accessory sheath comprising an elongated member having an accessory sheath body portion that includes an accessory sheath proximal end, an accessory sheath distal end opposite from the accessory sheath proximal end, and an accessory sheath lumen extending through the accessory sheath between the accessory sheath proximal end and the accessory sheath distal end, the accessory sheath including a curved section that is nearer the accessory sheath distal end than the accessory sheath proximal end, the curved section having a default shape and a first stiffness; and a dilator comprising an elongated member having a dilator proximal end and a dilator distal end opposite from the dilator proximal end, at least a portion of the dilator having a second stiffness that is greater than the first stiffness; where the dilator is configured to be disposed in the accessory sheath lumen with the portion of the dilator in the curved section of the accessory sheath such that dilator alters the shape of the curved section of the accessory sheath relative to the default shape.

Some embodiments of the present delivery systems for accessing a blood vessel comprise: an accessory sheath comprising an elongated member having an accessory sheath body portion that includes an accessory sheath proximal end, an accessory sheath distal end opposite from the accessory sheath proximal end, and an accessory sheath lumen extending through the accessory sheath between the accessory sheath proximal end and the accessory sheath distal end, the accessory sheath including a curved section that is nearer the accessory sheath distal end than the accessory sheath proximal end, the curved section defining a default shape, at least a portion of the accessory sheath comprising a shape memory material (SMM) defining the default shape at a temperature between at least 96 and 101 degrees Fahrenheit; where, at one or more temperatures between 60 and 80 degrees Fahrenheit, the SMM is configured to alter the shape of the curved section relative to the default shape.

Some embodiments of the present methods of positioning a catheter introducer system in a blood vessel, comprise: advancing the accessory sheath of an embodiment of the present delivery systems through a patient's blood vessel to a position at which the accessory sheath distal end is disposed in the patient's heart.

Some embodiments of the present methods of positioning a catheter introducer system in a blood vessel, comprise: advancing an accessory sheath of an embodiment of the present delivery systems, with a dilator disposed in the accessory sheath lumen, through a patient's blood vessel to a position at which the accessory sheath distal end is disposed in the patient's heart; withdrawing the dilator from the accessory sheath lumen to permit the curved section to return to the default shape; and rotating the accessory sheath until the accessory sheath distal end is substantially perpendicular to the atrial septum or atrial septal plane (a plane that is substantially parallel to the atrial septum) of the patient's heart.

Any embodiment of any of the apparatuses, systems, and methods can consist of or consist essentially of rather than comprise/include/contain/have any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

### BRIEF DESCRIPTION OF THE DRAWINGS

The accompanying drawings are included to provide a further understanding of the present disclosure and are incorporated in and constitute a part of this specification, illustrate embodiments described herein, and together with the description, serve to explain the principles discussed in this disclosure.

FIG. 1 is a side view of an example delivery system for accessing a blood vessel, in accordance with various aspects of the present disclosure;

FIG. 2 is a side view of an example introducer sheath that may be used with a delivery system for accessing a blood vessel, in accordance with various aspects of the present disclosure;

FIG. 3 is an enlarged side view of an example dilator that may be used with a delivery system for accessing a blood vessel, in accordance with various aspects of the present disclosure;

FIG. 4 is a side view of an example accessory sheath that may be used with a delivery system for accessing a blood vessel, in accordance with various aspects of the present disclosure;

FIG. 5 is a cutaway end view of the accessory sheath, shown in FIG. 4, in accordance with various aspects of the present disclosure;

FIG. 6 is an enlarged side view of a distal end portion of the accessory sheath, shown in FIGS. 4-5, in accordance with various aspects of the present disclosure;

FIG. 7 is an enlarged side view of a system for accessing a blood vessel with an example device delivery catheter deploying an occluder, in accordance with various aspects of the present disclosure;

FIG. 8 is a cutaway view of a human heart with a distal end view of the system, shown in FIG. 7, deploying an occluder in the atrial septum, in accordance with various aspects of the present disclosure; and

FIG. 9 is an example block flow diagram 900 illustrating a method for accessing a blood vessel, in accordance with various aspects of the present disclosure.

### DETAILED DESCRIPTION

Persons skilled in the art will readily appreciate that various aspects of the present disclosure can be realized by any number of methods and apparatuses configured to perform the intended functions. Stated differently, other methods and apparatuses can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Embodiments provided herein relate to catheter introducer systems that allow access to certain structures within the circulatory system of a patient. For example, some of the present embodiments are configured to allow access to the right atrium of a patient's heart to deliver an occluder or other therapy, such as to repair or treat an atrial septal defect (ASD). Some such embodiments are sized for children, while others are sized for adults, and may be sized for individuals of certain sizes (e.g., having a height of 3 feet to 4 feet, 4 feet to 5 feet, and/or the like). Others of the present embodiments can be configured to allow access to other structures or portions of the circulatory system of a patient. At least some of the present embodiments utilize an accessory sheath that is an elongated tubular member defining a default shape with one or more curves, the curvature of which may: (i) be altered (e.g., reduced) to facilitate insertion and/or removal of the accessory sheath into and along a patient's blood vessel to a desired location; and (ii) be restored (at least partially) at a desired location to facilitate a procedure to be performed at desired location (e.g., insertion of a distal end of the accessory sheath into a patient's right atrium to implant an occluder to treat an ASD or into a patient's left atrium via inter-atrial communication, such as through an existing iatrogenic ASD, for left atrial appendage closure, and/or valve therapy).

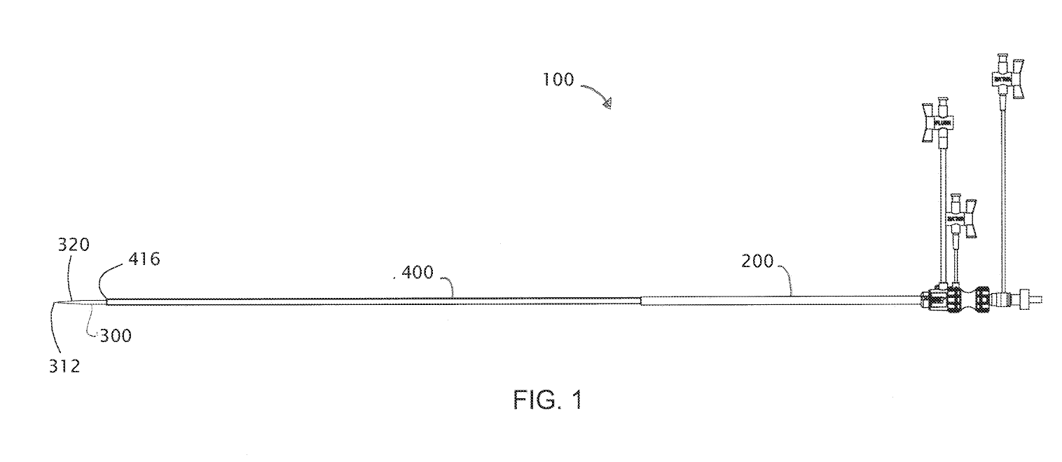

FIGS. 1-8 show and discuss various aspects of a delivery system for accessing a blood vessel, in accordance with various aspects of the present disclosure. The various shown and discussed system 100 may include an introducer sheath 200, a dilator 300, and an accessory sheath 400.

FIG. 1 depicts an example system 100 in an assembled state in which the dilator 300 is disposed in the accessory sheath 400 and the accessory sheath 400 is, in turn, disposed in the introducer sheath 200. The system may be used for accessing a blood vessel, in accordance with various aspects of the present disclosure.

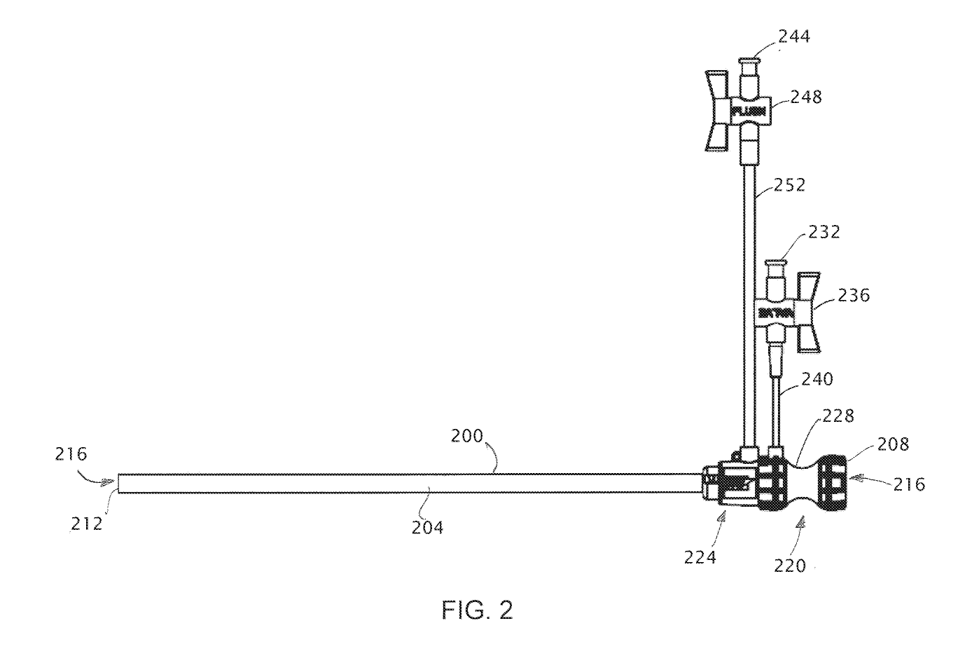

FIG. 2 is a side view of an example introducer sheath 200 that may be used with a delivery system for accessing a blood vessel, in accordance with various aspects of the present disclosure. As shown in FIG. 2, the introducer sheath 200 may include an elongated member 204 having an introducer sheath proximal end 208, an introducer sheath distal end 212 opposite from the introducer sheath proximal end 208, and an introducer sheath lumen 216 extending through the introducer sheath between the introducer sheath proximal end 208 and the introducer sheath distal end 212. The introducer sheath lumen 216 defines a diameter that is operable to slidingly receive the accessory sheath 400 therein and to allow the accessory sheath 400 to advance within the introducer sheath lumen 216. For example, in some embodiments, the introducer sheath lumen has an inner diameter of between substantially 1 mm to substantially 10 mm (e.g., between substantially 4 mm to substantially 8 mm). In this embodiment, the introducer sheath 200 further includes an inflatable lumen valve 220 and a flush-port equipped hub 224 coupling valve 220 to elongated member 204 at or adjacent introducer sheath proximal end 208.

As shown, the lumen valve 220 includes an annular seal 228 that is actuatable via a Luer port 232. More specifically, injection of fluid (an incompressible such as saline) or air into valve 220 via the Luer port 232 expands the annular seal 228 to close the entirety of introducer sheath lumen 216, or to close the portion of the introducer sheath lumen surrounding a dilator 300 (or device delivery catheter, described below, or other member or tool) disposed within the introducer sheath lumen 216. In certain instances, the dilator 300 may run through the accessory sheath lumen 416, and the annular seal 228 closes around the accessory sheath 400 to create a seal. Conversely, withdrawal of fluid from the annular seal 228 via the Luer port 232 contracts the annular seal 228 to open a portion of the introducer sheath lumen 216. In certain instances, the lumen valve 220 further includes a stopcock valve 236 disposed between and in fluid communication with the annular seal 228 (e.g., via tubing 240) and the Luer port 232 to selectively permit or prevent fluid communication between the annular seal 228 and the Luer port 232 to maintain a desired state of the annular seal 228. To maintain the annular seal 228 in an expanded, sealed state, for example, the stopcock valve 236 can be closed to prevent fluid from exiting the annular seal 228. Likewise, to maintain the annular seal 228 in a contracted, unsealed state, the stopcock valve 236 can be closed to prevent additional fluid from entering the annular seal 228.

As shown, the hub 224 may include a Luer port 244 through which liquid can be injected into or withdrawn from the introducer sheath lumen 216, such as, for example, to flush the introducer sheath lumen 216. In certain embodiments, the flush fixture 224 also includes a stopcock valve 248 between and in fluid communication with the introducer sheath lumen 216 (via tubing 252) and the Luer port 244 to selectively permit or prevent fluid communication between the introducer sheath lumen 216 and the Luer port 244.

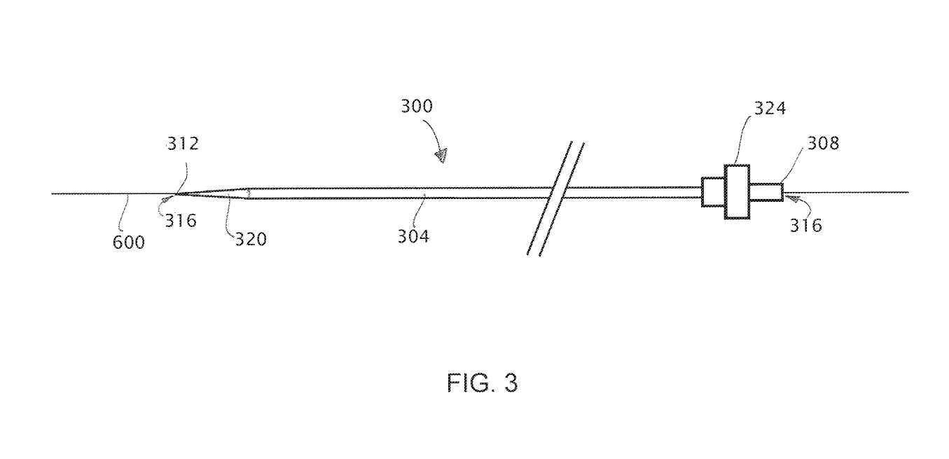

FIG. 3 is an enlarged side view of an example dilator 300 that may be used with a delivery system for accessing a blood vessel, in accordance with various aspects of the present disclosure. As shown in FIG. 3, the dilator 300 includes an elongated member 304 having a dilator proximal end 308 and a dilator distal end 312 opposite from the dilator proximal end 308. In certain instances, the dilator 300 also includes a dilator lumen 316 extending through the elongated member 304 through and between the dilator proximal end 308 and the dilator distal end 312. By way of example, in some embodiments, the dilator lumen 316 has a diameter of between substantially 0.038 inch to substantially 0.05 inch (e.g., a diameter large enough to receive a 0.035-inch guidewire). In other instances, however, the dilator lumen 316 may be omitted. The dilator lumen 316 defines a diameter that is operable to slidingly receive a guidewire 600 therein and to allow the dilator 300 to advance over the guidewire 600 (e.g., comprising an elongated member 604 that is operable for traversing blood vessels).

A guidewire 600 may be used to initially traverse a blood vessel, in accordance with various aspects of the present systems and methods. In general, a guidewire distal end of the guidewire 600 may be advanced through the blood vessel to a desired location. A relatively floppy guidewire distal end provides the ability for the guidewire 600 to be advanced through complex bends of a blood vessel. The dilator distal end 312 of the dilator 300 may be advanced over a guidewire proximal end and advanced through the blood vessel guided by the guidewire 600. During use, the guidewire 600 may be withdrawn and inserted or reinserted into the dilator lumen 316.

In certain instances, the dilator distal end 312 may include a dilator distal tip 320. The dilator distal tip 320 may be an integral element of the dilator distal end 312 or an element that is coupled to the dilator distal end 312. In some embodiments, the dilator distal tip 320 has a length of between substantially 1 cm to substantially 10 cm (e.g., between substantially 3 cm to substantially 8 cm, or between substantially 4 cm to about substantially 7 cm). The dilator distal tip 320 may or may not be tapered, but in most embodiments, a taper is provided on the outer diameter of the dilator distal tip 320 so that blood vessels are not exposed to steps and edges. Such a taper allows for, among other things, the dilator distal end 312 to have a reduced relative stiffness as compared with the non-tapered portion of the elongated member 304.

The dilator 300 may be formed by any suitable process, such as, but not limited to extrusion. The dilator distal tip 320 may be formed by any suitable process, such as, but not limited to, injection molding. In embodiments in which the elongated member 304 and the dilator distal tip 320 of the dilator are not unitary, the dilator distal tip 320 and elongated member 304 may be joined by any suitable process, such as, but not limited to thermal and chemical bonding. Alternatively, the dilator distal tip 320 may be unitary on the elongated member 304 of the dilator 300 as a one-piece component by any suitable process, such as, but not limited to grinding.

The dilator 300 is operable to dilate (enlarge) narrow portions of a blood vessel for the purpose of, for example, but not limited to, ensuring that the blood vessel may accept a catheter or sheath therein. In operation, in accordance with at least some embodiments, the dilator distal end 312 is advanced through the blood vessel. At narrow portions of the blood vessel, the dilator 300 (e.g., the dilator distal tip 320) comes into urging engagement with and enlarges the diameter of the blood vessel.

In some embodiments, the dilator 300 may include one or more radiopaque marker(s), such as, for example, an element that is coupled to the dilator 300 or by integrating one or more doping material(s) (e.g., barium sulfate) into the construct so as to assist with imaging by x-ray techniques, for example.

In certain instances, at least a portion (e.g., a distal portion) of the elongated member 304 of the dilator 300 has a stiffness that is greater than a corresponding portion of the accessory sheath 400 such that the dilator 300 can alter a shape (e.g., curvature) of a corresponding portion of the accessory sheath 400 when disposed therein, as described in more detail below. In certain instances (e.g., as shown in FIG. 1 with the introducer sheath 200 and in FIG. 3 with the guidewire 600), the introducer sheath 200 and the guidewire 600 are optional components that need not be supplied with the dilator 300 and/or the accessory sheath 400, and the accessory sheath 400 may be used with or without the introducer sheath 200 and guidewire 600. In other instances, a guidewire 600 will be used with or as part of the system including a dilator 300 and accessory sheath 400. In certain instances, a portion or portions of the dilator 300 may include a stiffness less than or greater than stiffness of other segments of the dilator 300. By controlling stiffness of the dilator 300 locally along the length of thereof, the dilator 300 may alter the shape of the accessory sheath 400 or the orientation of the dilator tip 320. In instances where the dilator tip 320 is relatively softer as compared to other portions of the dilator 300, the more flexible dilator tip 320 may help enhance enable access to more tortuous anatomies.

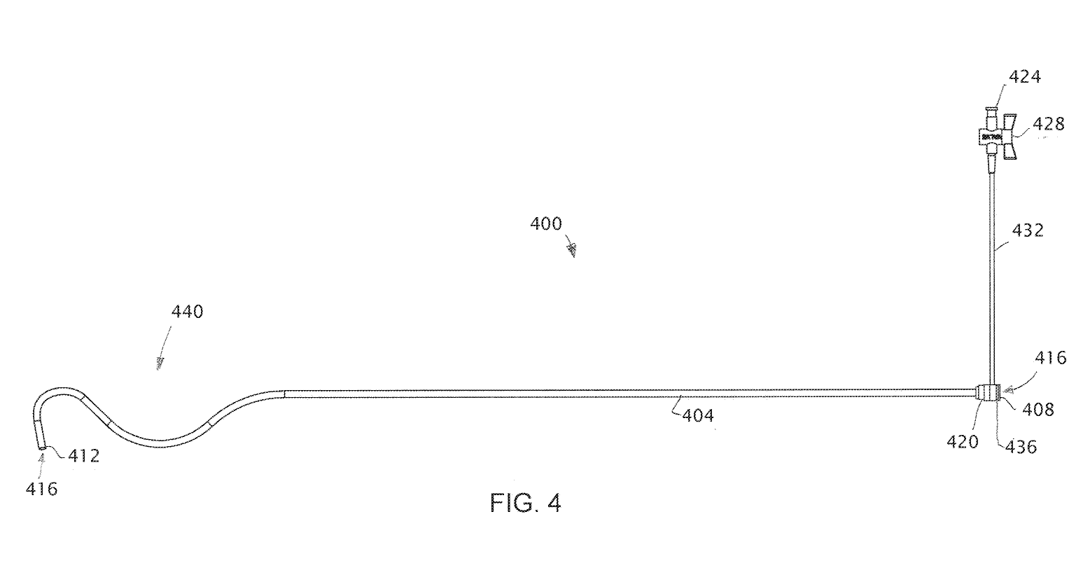

In certain instances, the dilator 300 may also include a hub 324 coupled to the elongated member 304 at or adjacent to the dilator proximal end 308. The hub 324 may be configured to couple the dilator 300 to the accessory sheath 400 (e.g., via keyed features of the hub 324 and of the accessory sheath 400). For example, the hub 324 can include one or more resilient locking tabs that can deflect and engage one or more corresponding grooves in a hub or flush fixture 420 of accessory sheath 400, (e.g., as shown in and described in further detail with reference to FIG. 4), when dilator 300 is fully disposed in the accessory sheath 400 with the dilator proximal end 308 adjacent to the accessory sheath proximal end 408 (as shown in FIG. 1) to resist separation of the dilator 300 and accessory sheath 400 and/or to resist relative rotation between the dilator 300 and accessory sheath 400. In other instances, the keyed features can include any suitable combinations of complimentary male/female ridge/groove features and/or the like. As shown, a length of the elongated member 304 of the dilator 300 is greater than a length of the accessory sheath lumen 416 such that, when fully disposed in the accessory sheath 400 with the dilator proximal end 308 adjacent to the accessory sheath proximal end 408, the dilator distal end 312 (e.g., the entirety of the tapered dilator distal tip 320) may extend out of and beyond the accessory sheath distal end 412 (e.g., as shown in FIG. 1). The dilator lumen 316 also extends through the dilator distal tip 320.

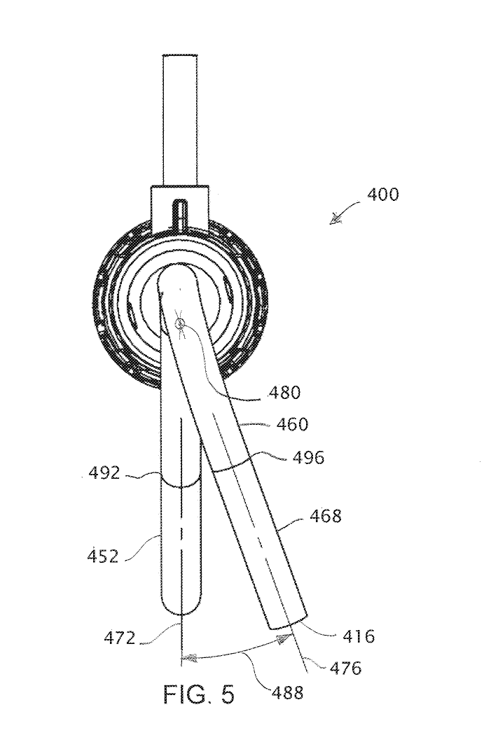

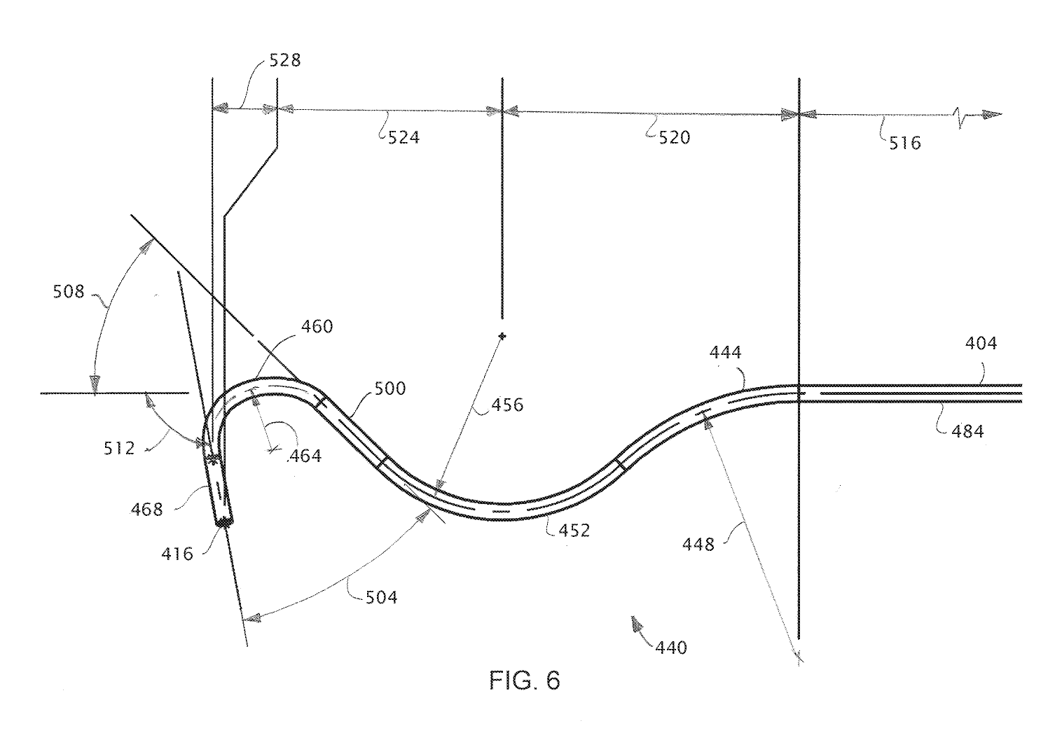

FIGS. 4-6 show different views of an example accessory sheath 400, in accordance with various aspects of the present disclosure. FIG. 4 is a side view of the accessory sheath 400 (that may be used with a delivery system for accessing a blood vessel), FIG. 5 is a cutaway end view of the accessory sheath 400, and FIG. 6 is an enlarged side view of a distal end portion of the accessory sheath 400, in accordance with various aspects of the present disclosure;

The accessory sheath 400 may include an elongated member having an accessory sheath body portion 404 that includes an accessory sheath proximal end 408, an accessory sheath distal end 412 opposite from the accessory sheath proximal end 408, and an accessory sheath lumen 416 extending through the accessory sheath 400 between the accessory sheath proximal end 408 and the accessory sheath distal end 412. In certain embodiments, the accessory sheath lumen 416 extends through at least one of (e.g., both of, as shown) the accessory sheath proximal end 408 and the accessory sheath distal end 412. The accessory sheath lumen 416 defines a diameter that is operable to slidingly receive the dilator 300 therein and to allow the dilator 300 to advance within the accessory sheath lumen 416. For example, in some embodiments, the accessory sheath lumen 416 has a diameter of between substantially 3 mm to substantially 6 mm, and/or is configured to slidingly receive a device-delivery catheter having a size of between 8 French to 18 French. In certain embodiments, the accessory sheath lumen 416 has a length (measured along the centerline of the accessory sheath 400) that is greater than a length of the introducer sheath lumen 216 such that, when fully disposed in the introducer sheath 200 with the accessory sheath proximal end 408 adjacent to the introducer sheath proximal end 208, the accessory sheath distal end 412 extends out of and beyond the introducer sheath distal end 216 (as shown in FIG. 1). As shown, the accessory sheath 400 includes a hub 420 having a proximal lumen seal 422 configured to restrict flow out of accessory sheath proximal end 408 while still permitting the dilator 300 to be inserted and removed through accessory sheath proximal end 408 (e.g.,, shown in FIG. 1). In this embodiment, the proximal lumen seal 422 may be an elastomeric sheet disposed across accessory sheath lumen 416 having one or more central slit(s) through which the dilator 300 can be inserted. In certain embodiments, the hub 420 comprises a Luer port 424 through which liquid can be injected into or withdrawn from the accessory sheath lumen 416, such as, for example, to flush the accessory sheath lumen 416. In addition, the hub 420 may also include a stopcock valve 428 between and in fluid communication with the accessory sheath lumen 416 (via tubing 432) and the Luer port 424 to selectively permit or prevent fluid communication between the accessory sheath lumen 416 and the Luer port 424. As shown, the hub 420 further defines an annular groove 436 configured to be engaged by a corresponding portion of the hub 324 of the dilator 300 to couple to the dilator 300 to the accessory sheath 400 together, such as is described above.

As shown in more detail in FIG. 6, the accessory sheath body portion 404 of the accessory sheath 400 may include a shaped or curved section 440 that is not straight or axial and that is nearer the accessory sheath distal end 412 than the accessory sheath proximal end 408. The curved section 440 can be shaped with or otherwise configured to adopt a default shape. For example, by way of structure (polymer alone, or polymer over a metallic wire with varying braid), material selection (e.g., polymers of varying mechanical properties), and/or manufacturing processes (e.g., molding or thermal shape-setting desired shapes, and/or reinforcing portions to retain particular shapes), curved section 440 can be configured with a first stiffness. In some embodiments, a default shape of the curved section 440 may be defined or altered by a user, such as, for example, by heating the curved section 440 (e.g., by submerging in water that has been heated) in excess of room temperature and/or normal human body temperature, defining or altering the desired default shape, and cooling the curved section while maintaining the defined default shape. As a result, the default shape can conform to the expected size and shape of a portion of a patient's circulatory system to facilitate access to that particular portion (e.g., a desired location) to deliver a therapeutic article such as an implantable medical device or therapeutic agent.

Various materials may be used to achieve the properties (e.g., default shape, stiffness, and/or the like) described herein for the accessory sheath 400. For example, in some embodiments, the accessory sheath body portion 404 can include a thermoplastic material (e.g., in extruded form) such as nylon or PEBAX® Polyether Block Amide copolymer (Arkema, Inc., King of Prussia, Pa.), and/or a metallic material (e.g., in coiled or braided wire form or tubular form) such as stainless steel or nickel titanium alloy (nitinol). Certain embodiments of the present accessory sheaths (such as the accessory sheath 400) further include one or more radiopaque marker(s) to assist visualization under x-ray imaging. Such radiopaque markers may, for example, be positioned at one or more of the accessory sheath distal end 412, and/or the curved section 440 of the accessory sheath 400.

Various embodiments of the present shaped accessory sheaths 400 can each include a curved section having any suitable shape. For example, such curved sections may be planar or in a three-dimensional configuration, may include one or more curved segments each defining a radius of curvature (e.g., between substantially 5 mm to substantially 100 mm), and/or may include one or more axial segments (e.g., each tangential to at least one adjoining curved segment), to define any shape suitable for disposing the accessory sheath distal end 412 in a desired relation to a structure within a patient's circulatory system. In some embodiments, each of one or more curved segments may not define a constant radius and may instead be compound curves of various radii on different planes.

In some embodiments of the present accessory sheaths, a majority of a curved section is disposed in a single plane, such a curved section may include portions disposed in two planes, such a curved section may include at least one straight or axial segment, and/or such a curved section may include at least one radius of curvature that is less than 125 mm. For example, the accessory sheath 400 is configured to be suitable for at least delivery of an occluder to the atrial septum to treat an ASD. In the embodiment shown, the default shape of the curved section 440 includes, from a proximal portion of the curved section 440 and extending toward a distal portion of the curved section 440 (e.g., toward accessory sheath distal end 412): a first curved segment 444 having a first radius of curvature 448; a second curved segment 452 with a second radius of curvature 456; and a third curved segment 460 with a third radius of curvature 464.

As shown in FIG. 6, for example, the second radius of curvature 456 is smaller than the first radius of curvature 448 and the curvature of the second curved segment 452 is in a different direction than the curvature of the first curved segment 444. Additionally, the third radius of curvature 464 is smaller than the second radius of curvature 456 and the curvature of the third curved segment 460 is in a different direction than the curvature of the second curved section 452. More specifically, and in certain instances, the first radius of curvature 448 may be between 2 inches to 4 inches (e.g., between 2.5 inches to 3.5 inches, between 2.75 inches to 3.25 inches, and/or substantially equal to 3 inches); the second radius of curvature 456 may be between 1 inch to 3 inches (e.g., between 1.5 inches to 2.5 inches, between 1.75 inches to 2.25 inches, and/or substantially equal to 2 inches); and third radius of curvature 464 may be smaller than 1 inch. In certain instances, the first radius of curvature 448 may be between 125% to 175% (e.g., between 140% to 160%, and/or substantially equal to 150%) of the second radius of curvature 456; and the second radius of curvature 456 may be more than 200% of the third radius of curvature 464.

In addition, the curved section 440 may also include: a first straight or axial segment 468 between the third curved segment 460 and the accessory sheath distal end 412. As shown in FIG. 5, the first curved segment 444 and the second curved segment 452 may be disposed in a first plane 472, and the first axial segment 468 is disposed in a second plane 476 that is rotated relative to the first plane around an axis 480 that extends parallel to at least a portion of the accessory sheath 400 between the accessory sheath proximal end 408 and the curved section 440. For example, the accessory sheath body portion 404 of the accessory sheath 400 may include a primary straight or axial (at least when in a relaxed, default state) segment 484 to which axis 480 is parallel. In certain embodiments, the second plane 476 is rotated relative to the first plane 472 by an angle 488, and the angle 488 is between 10 degrees to 30 degrees (e.g., between 15 degrees to 25 degrees, and/or substantially equal to 20 degrees). Further, the curved section 440 may transition between the first plane 472 and the second plane 476 along the third curved segment 460. More particularly, a first end 492 of the third curved segment 460 may be disposed in the first plane 472, and a second end 496 of the third curved segment 460 is disposed in the second plane 476.

Further yet, the curved section 440 further may include a second axial segment 500 disposed between the second curved segment 452 and the third curved segment 460. In addition, the second axial segment 500 may be angled relative to the first axial segment 468 by a first angle 504, and is angled relative to primary axial segment 484 by a second angle 508. As shown, the first angle 504 is substantially equal to 35 degrees; and the second angle 508 is substantially equal to 44 degrees; which results in an angle 512 between the first axial segment 468 and the primary axial segment 484 of substantially equal to 101 degrees.

As shown in FIG. 6, the accessory sheath 400 may have an overall length (measured along the centerline of the accessory sheath body portion 404) of substantially 25.6 inches (65 mm). In certain embodiments, a length 516 between the accessory sheath proximal end 408 and the most-proximal portion of the first curved segment 444 is substantially 16.7 inches (42.4 cm); a first distance 520 from the most-proximal portion of the first curved segment 444 to the center of the second curved segment 452 is substantially 3.8 inches (9.6 cm); a second distance 524 (measured parallel to the centerline of the primary axial segment 484) from the center of the second curved segment 452 to the centerline of the accessory sheath distal end 412 is substantially 3.2 inches (8.1 cm); and a third distance 528 (measured parallel to the centerline of the primary axial segment 484) from the centerline of the accessory sheath distal end 412 to the most-distal point on the centerline of the accessory sheath body portion 404 (i.e., within third curved segment 460, in this embodiment) is substantially 0.14 inches (0.35 cm). In certain embodiments, the overall length (measured parallel to the centerline of the primary axial segment 484) of the curved section 440 (the sum of distances 520, 524, and 528) is substantially between 30% to 50% (e.g., between 35% to 45%, and/or substantially equal to 40%) of length 516 of the primary axial segment 484.

In certain embodiments, the dilator 300 is configured to be disposed in the accessory sheath lumen 416 such that dilator 300 alters the shape of the curved section 440 of the accessory sheath 400 (e.g., FIG. 1) relative to a default shape of the accessory sheath 400 (e.g., FIG. 4). For example, a portion of the dilator 300 corresponding to (disposed within) the curved section 440 when the dilator 300 may be fully inserted into the accessory sheath (as shown in FIG. 1), may have a stiffness that is greater than a stiffness of the curved section 440 of the accessory sheath 400 such that the curved section 440 will tend to conform to the shape of the dilator. FIGS. 1, 3, and 4 illustrate one example such example. In particular, the dilator 300 may have a shape that is different than that of the curved section 440 of the accessory sheath 400 (e.g., straight, as shown). However, the stiffness of the dilator 300 (or at least the portion of dilator 300 corresponding to the curved section 440 of the accessory sheath 400) may be greater than the stiffness of the curved section 440 to such a degree that insertion of the dilator 300 into the accessory sheath lumen 416 alters the shape of the accessory sheath 400 (e.g., of at least the curved section 440) relative to the default shape, such as, for example, by reducing the curvature (i.e., increasing the radius of curvature of at least one curve and/or effectively increasing the length of an axial segment by straightening a portion of a curve) of relative to that of the default shape. For example, the dilator 300 may be configured to straighten the curved section 440 (FIG. 1) to facilitate insertion of the accessory sheath distal end 412 into a desired location in the circulatory system of a patient. Once the accessory sheath distal end 412 is at or near the desired location, or as the accessory sheath distal end 412 is advanced toward the desired location, the dilator 300 can be removed from the accessory sheath lumen 416 to allow the curved section 440 to return (e.g., resiliently) to its default shape.

In other embodiments, the accessory sheath 400 (e.g., at least the curved section 440) is shaped with or otherwise configured to adopt its default shape by a shape memory material (SMM) included in the accessory sheath body portion 404. For example, in some embodiments, a nitinol wire or nitinol hypotube is embedded within the accessory sheath body portion 404 (e.g., at least the curved section 440) to bias the curved section 440 toward the default shape in at least some conditions (e.g., at certain temperatures). In some embodiments, for example, a shape memory material (e.g., nitinol) is configured to define the default shape at least one temperature between 96 and 101 degrees Fahrenheit, and to alter the default shape or permit the default shape to be more-readily altered (e.g., have a lower stiffness relative to the stiffness at the temperature between 96 and 101 degrees Fahrenheit) at least one temperature between 60 and 80 degrees Fahrenheit. In such embodiments, the accessory sheath 400 (e.g., at least the curved section) can adopt a lower stiffness and/or alter the shape of the curved section 440 relative to the default shape dependent on temperature. For example, the SMM can cause the curved section 440 to adopt a second shape (e.g., having relatively less curvature or larger radii of curvature than the default shape). In such embodiments, the accessory sheath 400 (e.g., at least the curved section 440) can be configured to have a relatively lower stiffness and/or adopt the second shape at room temperature to facilitate insertion of the accessory sheath distal end 412 to a desired location within a patient, and to adopt the default shape after insertion as the accessory sheath 400 warms to the body temperature of the patient.

In addition and in certain instances, the dilator 300 may comprise a material with a high degree of thermal conductivity, such as a stainless steel or NiTi. The dilator 300 may be chilled to temporarily decrease the bulk phase temperature of the accessory sheath 400 to a point where it begins to transition out of its preferred high-temp geometry into something less stiff and less curved for ease of removal. This dilator 300 may be chilled by wiping the dilator 300 with isopropyl alcohol and allowing the evaporative cooling effect to lower the bulk phase temperature of the dilator 300 prior to insertion into the accessory sheath 400.

In certain instances, the accessory sheath 400 may be arranged in its default shape prior to insertion in to the body. The dilator 300 may be stiff and disposed at least partially within the accessory sheath lumen 416. The accessory sheath 400 may curve as it is pulled over the dilator 300 (stress-induced martensite may occur and removal thereof may become easier).

Other properties of a shape memory material may also be useful in certain instances. In certain embodiments, for example, a pseudoelastic property of a nickel-titanium alloy, for example, provides that when under the stress of the dilator 300 inserted into the accessory sheath 400, a curvature of the accessary sheath 400 will straighten by way of stress induced martensite. Removal of the dilator 300 will remove the induced stress which results in the accessory sheath 400 returning back to a default shape.

In certain embodiments, the maximum stiffness of the assembled dilator 300 and accessory sheath 400 is preferably small or low enough to both avoid damaging blood vessels encountered during expected use (e.g., for a particular procedure) and to permit the assembly to be guided within bends of blood vessels encountered during such an expected use. Likewise, in certain embodiments, the maximum stiffness of the accessory sheath 400 alone is preferably large enough to bias the curved section 440 of the accessory sheath 400 to the default shape, but small or low enough to both avoid damaging blood vessels encountered during expected use (e.g., for a particular procedure) and to permit the assembly to be guided within bends of blood vessels encountered during such an expected use.

Some embodiments of the present systems or kits can comprise a number of accessory sheaths (e.g., 400) each defining a different curved section. For example, a system or kit of the present accessory sheaths for a particular procedure (e.g., repair of an ASD) can include a plurality of accessory sheaths with respective curved sections that are similar in overall configurations but sized for variations in sizes within a group of patients (e.g., children, adolescent males adult females, and/or the like). The number and type of accessory sheaths included in such a kit may depend on an intended procedure (e.g., due to the particular location in a patient's circulatory system at which the procedure is to be performed and/or the path taken within the body to arrive at such a particular location).

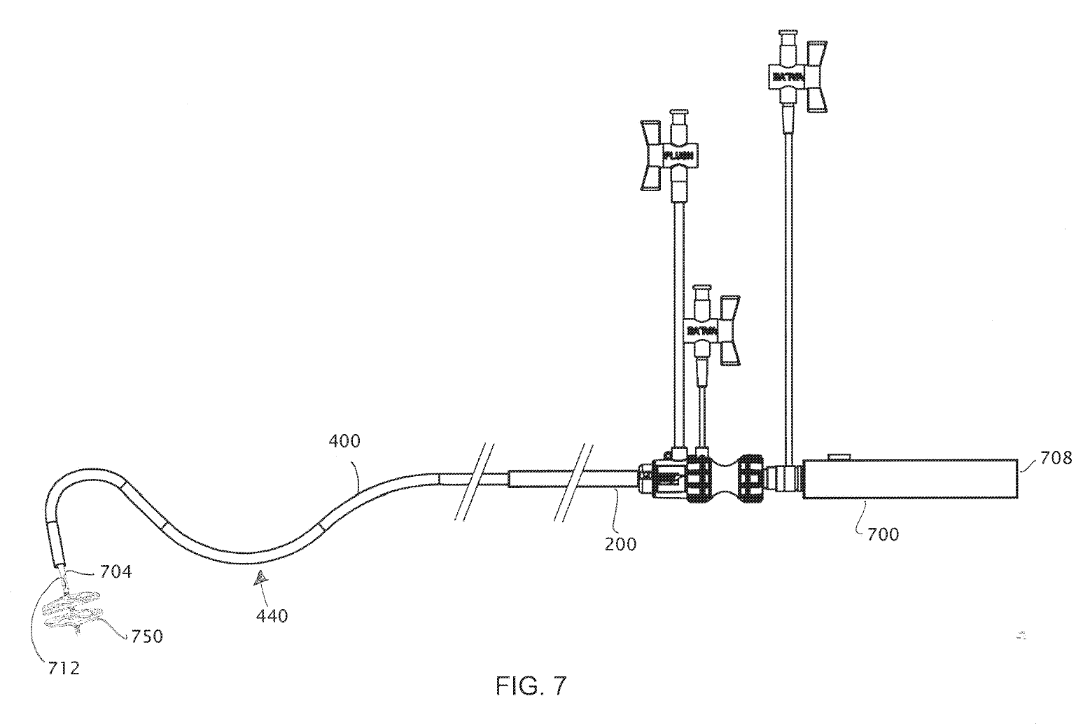

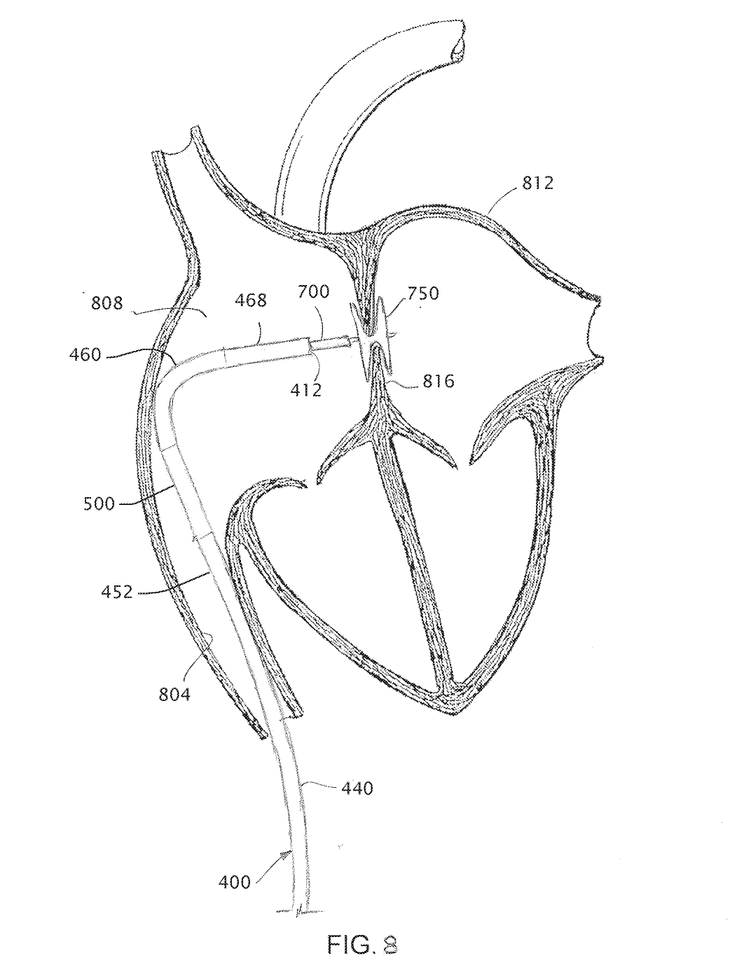

FIGS. 7 and 8 show views of an example system for accessing a blood vessel with a device delivery catheter 700 deploying an occluder, in accordance with various aspects of the present disclosure. Referring now to FIGS. 7-8, shown therein is the device-delivery catheter 700 disposed within an accessory sheath lumen 416 of the accessory sheath 400. In the embodiment shown, the device-delivery catheter 700 includes an elongated member having a catheter body 704 that includes a catheter proximal end 708 and a catheter distal end 712 opposite from the catheter proximal end 708. In this embodiment, the device-delivery catheter 700 (or at least a portion thereof that corresponds to that portion that is disposed within the curved section 440 of the accessory sheath 400) may have a stiffness that is lower than the stiffness of the curved section 440 such that the device-delivery catheter 700 can be disposed in the accessory sheath lumen 416 with the catheter distal end 712 extending beyond the accessory sheath distal end, as shown (e.g., without substantially altering the default shape).

As illustrated in FIG. 8, the curved section 440 of the accessory sheath 400 defines a shape that is adapted to support the device-delivery catheter 700 to track at least one of a bend in a blood vessel (e.g., inferior vena cava 804) and portion of an organ (e.g., right atrium 808 of the heart 812) in the patient's circulatory system. More particularly, the curved section 440 may be configured to track the depicted portion of the patient's inferior vena cava 804 and right atrium 808 in such a way that the accessory sheath distal end 412 (and first axial segment 468) are substantially perpendicular to the atrial septum 816 to facilitate effective installation of an occluder 750 as shown.

For example, for at least the relative sizes of accessory sheath 400 and heart 812, the third curved segment 460 of the accessory sheath 400 may contact an inner surface of the heart that defines the right atrium 808, and/or the second curved segment 452 may contact an inner surface of the inferior vena cava to assist with stabilizing the first axial segment 468 in an orientation in which the first axial segment 468 (and accessory sheath distal end 412) is substantially perpendicular to the atrial septum 816. In some embodiments, the accessory sheath 400 may be sized such that the second curved segment 452 of the accessory sheath 400 contacts the inferior vena cava 804 in two places to further stabilize the accessory sheath. In some embodiments, the curved section 440 of the accessory sheath 400 can also modify the vasculature pathway to the heart to position a device in a more opportune orientation relative to the septum.

The described orientation in which distal end 412 (and first axial segment 468) of the accessory sheath 400 permits more accurate and effective placement of an occluder 750. For example, at least some occluders 750 may be expected to seal more reliably and/or more effectively when inserted and/or expanded at such an orientation (with a longitudinal axis perpendicular to the atrial septum 816). As described above, the present accessory sheaths 400 are therefore configured to facilitate the deployment of an occluder 750 at such an orientation, and may further be configured to improve the stability of the distal end 412 (and first axial segment 468) of the accessory sheath 400 during deployment of the occluder.

In certain instances, the curved sections of the accessory sheath 400 may create space in the right atrium 808 during deployment of the occluder 750. Particularly in smaller patients with limited atrial chamber volume, or in patients where the IVC ostium is relatively closely positioned to the atrial septum 816, the lack of space to unfurl and form the occluder 750 (and more particularly a right disc of the occluder within the right atrium 808) may lead to prolapse of the occluder 750 out of the defect. Incorporation of the curvature (such as the curved segment 460) of the accessory sheath 400 may serve to create the additional space necessary to deploy the right disc of the occluder 750 without pushing a portion of the disc through the defect and into the left atrium. The curvature may also allow for the occluder 750 to be advanced of the accessory sheath 400 into the left atrium may occur to deploy the left disc of the occluder 750. In certain instances, the orthogonal approach may result in the left disc being parallel with the plane of the atrial septum 816. This sequence may allow for retraction of the delivery system 100 into the right atrium 808 to complete the deployment sequence without moving the position of the accessory sheath 400.

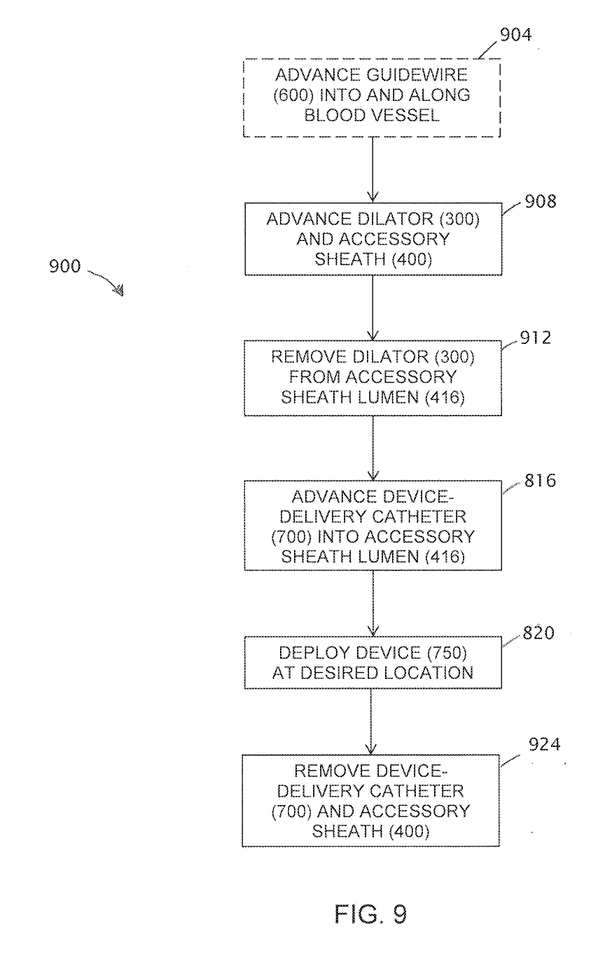

FIG. 9 is an example block flow diagram 900 illustrating an example method for accessing a blood vessel, in accordance with various aspects of the present disclosure. As is shown at block 904, the method may include an optional step of advancing a guidewire (e.g., 600) into and along a blood vessel. As is shown at block 908, the method may include advancing a dilator (e.g., 300) and shaped accessory sheath (e.g., 400) into and along the blood vessel (e.g., over the guidewire), such as, for example, to a position at which the accessory sheath distal end is disposed in the patient's heart. As is shown at block 912, the method may include removing the dilator from the accessory sheath lumen (e.g., 416). Before or after the dilator is removed, certain methods may include rotating the accessory sheath until the accessory sheath distal end is in a desired orientation (e.g., substantially perpendicular to the atrial septum of the patient's heart). As is shown at block 916, the method may also include advancing a device-delivery catheter (e.g., 700) through the patient's blood vessel via the accessory sheath lumen. As is shown at block 920, the method includes deploying a device (e.g., 750) from the device-delivery catheter (e.g., installed in an ASD). As is shown at block 924, the method includes removing the device-delivery catheter and accessory sheath (and guidewire, if applicable).

As used herein, the term “couple” means to join, connect, attach, adhere, affix, or bond, whether directly or indirectly, and whether permanently or temporarily.

As used herein, the term “substantially” is defined as largely but not necessarily wholly what is specified (and includes what is specified; e.g., substantially 90 degrees includes 90 degrees and substantially parallel includes parallel), as understood by a person of ordinary skill in the art, that is within a range that is suitable to bring about the intended purpose or function.

The preposition “between,” when used herein to define a range of values (e.g., between x and y) means that the range includes the end points (e.g., x and y) of the given range and the values between the end points.

As used herein, the terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements, but is not limited to possessing only those elements. Likewise, a method that “comprises,” “has,” “includes,” or “contains” one or more steps possesses those one or more steps, but is not limited to possessing only those one or more steps.

As used herein, “blood vessel” refers to not only an element of the vasculature, but any blood conduit of the cardiovascular system, including the heart.

As used herein, a “tortuous” blood vessel refers to a blood vessel that is particularly difficult to advance an intraluminal device through usually due to tight and/or reverse bends defined by the path of the blood vessel. An example of a pathway that involves particularly tortuous blood vessels is transvenous access to the main pulmonary artery from the femoral vein.

As used herein, “dilator” refers to an elongated tubular member that may be used to enlarge or stretch a body part, such as a blood vessel, cavity, canal, or orifice. A dilator may be used to introduce and guide a sheath into and through a blood vessel. A dilator may be relatively less stiff than a sheath in accordance with constructs presented therein.

As used herein, “lumen” refers to a longitudinal cavity or through-bore along a longitudinal axis in a tubular component. A lumen may extend partially, completely, or substantially the axial length of a component, such as, for example, a dilator, sheath, or core.

As used herein, “assembly” or “introducer assembly” refers to two or more components of a catheter introducer system, such as combinations of dilators, sheaths, guidewires, and/or cores that are engaged or coupled.

As used herein, “shaped” refers to a component that is formed to a predetermined pattern, geometry, curvature or angle. Accessory sheaths, cores, and/or dilators may be shaped to conform to or follow a particular pattern, geometry, curvature, or angle. Such sheaths, cores, and dilators each may include curves in one or more planes and/or in three-dimensional configurations. Dilators and/or cores may also be straight and used only to impart a desired level of stiffness to the system or portion of the system (e.g., an assembly of an accessory sheath and a dilator).

As used herein, “core” refers to an elongated tubular member capable of being received within a dilator lumen so as to impart a desired shape and/or stiffness to the dilator. A core can be manufactured of any material possible of forming and supporting a curve, such as, but not limited to, a metal, thermoplastic or molded or cast thermoset material. Nylon is an example of a material that may be used to form a core. Stainless steel and nitinol are examples of metals that may be used to form a core.

As used herein, “sheath” refers to an elongated tubular member operable for providing a guide way or conduit for introducing medical devices such as catheters into the body. The sheath may be positioned within the body with the assistance of a dilator. The present shaped accessory sheaths can be manufactured of any material possible of forming and supporting a curve, such as, but not limited to, a metal, thermoplastic, or molded or cast thermoset material. Nylon is an example of a material that may be used to form such a sheath. Stainless steel and nitinol are examples of metals that may be used to form such a sheath.

As used herein, “curve” and “curvature” refer to a shape, geometry or radius of a component, such as a core or dilator. The curve of a component may be formed in one or more planes or in three-dimensional configurations and each component (e.g., dilator or core) can include none (straight), one curve, and/or more than one curve.

As used herein, “bend” refers to a change of direction of the path defined by a blood vessel, organ or other body cavity.

As used herein, “stiffness” refers to the property of a component to resist bending. A dilator and/or a core may be used to provide a system with increased stiffness as compared with the stiffness of the individual components making up the system. For example, disposing a relatively stiff dilator within a sheath lumen of a curved accessory sheath will provide a dilator/accessory sheath assembly with a higher stiffness than the accessory sheath itself.

As used herein, “guidewire” refers to a wire or small diameter elongated member that may be advanced through a blood vessel or cavity of the body.

As used herein, “distal” refers to a region or location positioned away from a point of origin or attachment.

As used herein, “proximal” refers to a region or location positioned adjacent or near a point of origin or attachment.

As used herein, “tapered” refers to a change in physical dimension along a length of a component.

As used herein, “elongated” refers to a region of extended length.

As used herein, “radiopaque marker” refers to an element that resists the passage of x-ray or other electromagnetic radiation for at least the purpose of monitoring positioning using x-ray techniques.

As used herein, “interchangeably” refers to two or more components that can replace one another in a similar position or function. For example, two or more cores may be interchangeable within a dilator to impart differing curves onto the dilator.

It will be apparent to those skilled in the art that various modifications and variations can be made in the present embodiments without departing from the spirit or scope of the embodiments. Thus, it is intended that the present embodiments cover the modifications and variations of this invention provided they come within the scope of the appended claims and their equivalents.

1. A delivery system for accessing a blood vessel, comprising:
an accessory sheath comprising an elongated member having an accessory sheath body portion that includes an accessory sheath proximal end, an accessory sheath distal end opposite from the accessory sheath proximal end, and an accessory sheath lumen extending through the accessory sheath between the accessory sheath proximal end and the accessory sheath distal end, the accessory sheath including a curved section that is nearer the accessory sheath distal end than the accessory sheath proximal end;
where a default shape of the curved section includes, from a proximal portion of the curved section and extending toward a distal portion of the curved section:
a first curved segment having a first radius of curvature;
a second curved segment with a second radius of curvature that is smaller than the first radius of curvature and the curvature of the second curved segment is in a different direction than the curvature of the first curve;
a third curved segment with a third radius of curvature that is smaller than the second radius of curvature and the curvature of the third curved segment is in a different direction than the curvature of the second curve; and
a first axial segment between the third curved segment and the accessory sheath distal end,
wherein the first curved segment and the second curved segment are disposed in a first plane, and the first axial segment is disposed in a second plane that is rotated relative to the first plane around an axis that extends parallel to at least a portion of the accessory sheath between the accessory sheath proximal end and the curved section.

2. The delivery system of claim 1, where the curved section further includes a second axial segment disposed between the second curved segment and the third curved segment.

3. The delivery system of claim 1, where a first end of the third curved segment is disposed in the first plane, and a second end of the third curved segment is disposed in the second plane.

4. The delivery system of claim 1, where the curved section has a first stiffness, and the delivery system further comprises:
a dilator comprising an elongated member having a dilator proximal end and a dilator distal end opposite from the dilator proximal end, at least a portion of the dilator having a second stiffness that is greater than the first stiffness,
where the dilator is configured to be disposed in the accessory sheath lumen with the portion of the dilator in the curved section of the accessory sheath such that dilator alters the default shape of the curved section.

5. A delivery system for accessing a blood vessel, comprising:
an accessory sheath comprising an elongated member having an accessory sheath body portion that includes an accessory sheath proximal end, an accessory sheath distal end opposite from the accessory sheath proximal end, and an accessory sheath lumen extending through the accessory sheath between the accessory sheath proximal end and the accessory sheath distal end, the accessory sheath including a curved section that is nearer the accessory sheath distal end than the accessory sheath proximal end, the curved section having a default shape and a first stiffness; and
a dilator comprising an elongated member having a dilator proximal end and a dilator distal end opposite from the dilator proximal end, at least a portion of the dilator having a second stiffness that is greater than the first stiffness,
wherein the dilator is configured to be disposed in the accessory sheath lumen with the portion of the dilator in the curved section of the accessory sheath such that dilator alters the default shape of the curved section.

6. The delivery system of claim 5, further comprising:
a device-delivery catheter comprising an elongated member having a catheter proximal end and a catheter distal end opposite from the catheter proximal end, the device-delivery catheter having a third stiffness that is lower than the first stiffness such that the device-delivery catheter can be disposed in the accessory sheath lumen with the catheter distal end extending beyond the accessory sheath distal end.

7. A delivery system for accessing a blood vessel, comprising:
an accessory sheath comprising an elongated member having an accessory sheath body portion that includes an accessory sheath proximal end, an accessory sheath distal end opposite from the accessory sheath proximal end, and an accessory sheath lumen extending through the accessory sheath between the accessory sheath proximal end and the accessory sheath distal end, the accessory sheath including a curved section that is nearer the accessory sheath distal end than the accessory sheath proximal end, the curved section defining a default shape, at least a portion of the accessory sheath comprising a shape memory material (SMM) defining the default shape at a temperature between at least 96 and 101 degrees Fahrenheit,
wherein, at one or more temperatures between 60 and 80 degrees Fahrenheit, the SMM is configured to alter the default shape of the curved section.

8. The delivery system of claim 7, further comprising:
a dilator comprising an elongated member having a dilator proximal end and a dilator distal end opposite from the dilator proximal end, the dilator configured to be disposed in the accessory sheath lumen such that the dilator distal end extends beyond the accessory sheath distal end.

9. The delivery system of claim 8, where the dilator includes a dilator lumen extending through the elongated member through and between the dilator proximal end and the dilator distal end.

10. The delivery system of claim 8, where a length of the dilator body is greater than a length of the accessory sheath lumen.

11. The delivery system of claim 1, further comprising:
an introducer sheath comprising an elongated member having an introducer sheath proximal end, an introducer sheath distal end opposite from the introducer sheath proximal end, and an introducer sheath lumen extending through the introducer sheath between the introducer sheath proximal end and the introducer sheath distal end.

12. The delivery system of claim 11, where a length of the accessory sheath is greater than a length of the introducer sheath lumen.

13. The delivery system of claim 1, where the accessory sheath lumen extends through the accessory sheath proximal end.

14. The delivery system of claim 13, where the accessory sheath lumen extends through the accessory sheath distal end.

15. The delivery system of claim 1, where the system is configured such that, during alteration of the default shape of the curved section, a curvature of at least one curve is reduced relative to that of the default shape.

16. The system of claim 1, where the curved section of the accessory sheath includes at least one straight segment.

17. The system of claim 1, where a majority of the curved section of the accessory sheath is disposed in a single plane.

18. The system of claim 1, where the curved section of the accessory sheath includes portions disposed in at least two planes.

19. The delivery system of claim 5, where the default shape of the curved section includes, from a proximal portion of the curved section and extending toward a distal portion of the curved section:
a first curved segment having a first radius of curvature;
a second curved segment with a second radius of curvature that is smaller than the first radius of curvature and the curvature of the second curved segment is different direction than the curvature of the first curve;
a third curved segment with a third radius of curvature that is smaller than the second radius of curvature and the curvature of the third curved segment is in a different direction than the curvature of the second curve; and
a first axial segment between the third curved segment and the accessory sheath distal end,
wherein the first curved segment and the second curved segment are disposed in a first plane, and the first axial segment is disposed in a second plane that is rotated relative to the first plane around an axis that extends parallel to at least a portion of the accessory sheath between the accessory sheath proximal end and the curved section.

20. The delivery system of claim 19, where the curved section further includes a second axial segment disposed between the second curved segment and the third curved segment.

21. The delivery system of claim 19, where a first end of the third curved segment is disposed in the first plane, and a second end of the third curved segment is disposed in the second plane.

22. The delivery system of claim 1, further comprising
a guidewire comprising an elongated member that is operable for traversing tortuous blood vessels,
wherein the dilator includes a dilator lumen extending through the dilator from the dilator proximal end to the dilator distal end, the dilator lumen is operable to receive the guidewire, and the dilator is operable to be advanced over the guidewire.

23. The delivery system of claim 1, further comprising a plurality of accessory sheaths, each defining a curved section that differs from the curved section of at least one other of the plurality of accessory sheaths.

24. The delivery system of claim 1, wherein the default shape of the curved section of the accessory sheath is adapted to support a device-delivery catheter to track a bend in a blood vessel.

25. The delivery system of claim 1, wherein the curved section of the accessory sheath includes at least one radius of curvature that is less than 125 mm.

26. The delivery system of claim 1, wherein the accessory sheath distal end may be shaped into the curve by a user.

27. The delivery system of claim 1, wherein the accessory sheath comprises one or more of a thermoplastic and metal.

28. The delivery system of claim 27, wherein the accessory sheath comprises nylon.

29. The delivery system of claim 27, wherein the accessory sheath comprises polyether block amide (PEBAX).

30. The delivery system of claim 27, wherein the accessory sheath comprises nitinol.

31. The delivery system of claim 27, wherein the accessory sheath comprises stainless steel.

32. The delivery system of claim 27, wherein the accessory sheath comprises one or more radiopaque markers to assist visualization under x-ray imaging.

33. The delivery system of claim 32, wherein the one or more radiopaque markers are positioned at one or more of the accessory sheath distal end and the curved section of the accessory sheath.

34. The delivery system of claim 1, where the accessory sheath lumen has a diameter of between 3 mm to 6 mm.

35. The delivery system of claim 34, where the accessory sheath lumen is configured to receive a device-delivery catheter having a size of 8 to 18 French.

36. The delivery system of claim 5, further comprising a hub coupled to the dilator proximal end and operable to couple the dilator to the accessory sheath.

37. The delivery system of claim 5, wherein the dilator comprises one or more radiopaque markers to assist visualization under x-ray imaging.

38. The delivery system of claim 36, wherein the hub is operable to couple the dilator to the accessory sheath via keyed features of the hub and of the accessory sheath.

39. The delivery system of claim 38, wherein the keyed features comprise complimentary male/female ridge/groove features.

40. The delivery system of claim 1, wherein the dilator has a stiffness that is large enough to bias the curved section of the accessory sheath to the default shape, but not so large as to damage the blood vessel or not be able to be guided within bends of the blood vessel.

41. The delivery system of claim 1, further comprising:
a hemostatic valve coupled to the accessory sheath proximal end and configured to close a part or all of the accessory sheath lumen at the accessory sheath proximal end.

42. A method of positioning a catheter introducer system in a blood vessel, comprising:
providing a delivery system of claim 1; and
advancing the accessory sheath through a patient's blood vessel to a position at which the accessory sheath distal end is disposed in the patient's heart.

43. A method of positioning a catheter introducer system in a blood vessel, comprising:
providing a delivery system of claim 4;
advancing the accessory sheath, with the dilator disposed in the accessory sheath lumen, through a patient's blood vessel to a position at which the accessory sheath distal end is disposed in the patient's heart;
withdrawing the dilator from the accessory sheath lumen to permit the curved section to return to the default shape; and
rotating the accessory sheath until the accessory sheath distal end is substantially perpendicular to a septum of the patient's heart.

44. A method of positioning a catheter introducer system in a blood vessel, comprising:
providing a delivery system of claim 8;
advancing the accessory sheath, with the dilator disposed in the accessory sheath lumen, through a patient's blood vessel to a position at which the accessory sheath distal end is disposed in the patient's heart;
withdrawing the dilator from the accessory sheath lumen; and
rotating the accessory sheath until the accessory sheath distal end is substantially perpendicular to a septum of the patient's heart.
